(19) United States
(12) Patent Application Publication
(10) Pub. No.: US 2008/0125634 A1
(43) Pub. Date: May 29, 2008

(54) METHOD AND APPARATUS FOR IDENTIFYING AND TREATING MYOCARDIAL INFARCTION

(75) Inventors: S. Eric Ryan, Hopkinton, MA (US); Jing Tang, Arlington, MA (US)

Correspondence Address: MILLS & ONELLO LLP, ELEVEN BEACON STREET, SUITE 605, BOSTON, MA, 02108, US

(73) Assignee: CORNOVA, INC., Burlington, MA (US)

(21) Appl. No.: 11/762,956

(22) Filed: Jun. 14, 2007

Related U.S. Application Data
(60) Provisional application No. 60/804,709, filed on Jun. 14, 2006.

(51) Int. Cl. A61B 18/20; A61B 5/1459; A61B 10/02; A61M 25/00

(52) U.S. Cl. 600/342; 600/562; 604/511; 606/15; 607/89

(57) ABSTRACT

A method and apparatus for analyzing and treating internal tissues and, in particular, tissues affected by myocardial infarct. The apparatus includes a catheterized device integrating an optical probe and treatment delivery system. The probe component includes fiber optic lines that can be used in conjunction with infrared spectroscopy to analyze various characteristics of tissues, including chemical, blood, and oxygen content, in order to locate those tissues associated with myocardial infarct, to determine the best location for applying treatment, and to monitor treatment and its effects. Physically integrated with the probe component is a treatment component for delivering treatments including stem cell and gene therapy, known for having beneficial effects on tissues associated with myocardial infarct. A control system coordinates operation of the catheter, including performing chemometric analysis with the use of model data, and for providing control and visual feedback to an operator.

### RELATED APPLICATIONS

This application claims the benefit of U.S. Patent Application No. 60/804,709, filed on 14 Jun. 2006, entitled “Method and Apparatus for Identifying and Treating Myocardial Infarction,” the contents of which is incorporated herein in its entirety by reference.

### FIELD OF THE INVENTION

This invention relates to methods and apparatus for identifying, localizing, and treating diseased internal tissues including myocardial infarctions which, in particular, employ catheters having optical-probe and needle-injection assemblies.

### BACKGROUND OF THE INVENTION

Cardiovascular diseases and disorders are the leading cause of death and disability in all industrialized nations. In the United States alone, an estimated 700,000 Americans suffered a stroke in 2005—that's at least one stroke victim every 45 seconds. Stroke is the No. 3 killer and a leading cause of severe, long-term disability in the United States. In 2005, the estimated direct and indirect costs of cardiovascular diseases and stroke were $393.5 billion (as reported by the American-Heart-Association).

One of the primary factors that render cardiovascular disease particularly devastating is the heart's inability to repair itself following damage. Since myocardial cells are unable to divide and repopulate areas of damage, cardiac cell loss as a result of injury or disease is largely irreversible. Myocardial necrosis may generally begin near the endocardial surface. Depending on a number of factors, including the location of the affected area, this necrosis may or may not progress into a transmural infarct. Over time, adjacent regions may become infarcted as well due to retrograde propagation of the thrombus, development of micro emboli, arrhythmias, or other similar factors, leading to infarcts arising at different times within the same affected area.

Although it is not generally possible to revive necrotic (or dead) myocardial tissue, promising new advances in stem cell and gene therapy for regenerating otherwise dead tissue are being realized. Myocardial cells that are key to proper operation of the heart include cardiomyocyte (muscle cells) for pumping blood and endothelial cells (vessel cells) for circulating blood and nutrients. Research studies suggest that directly injecting certain types of primitive cells (e.g. stem cells, bone marrow) in areas surrounding necrotic cardiomyocyte cells (e.g. periinfarct areas) can induce regeneration of the dead myocardial tissue. See Stem Cells: Scientific Progress and Future Research Directions. Department of Health and Human Services. June 2001; retrieved from the Internet: <URL:http://stemcells.nih.gov/info/scireport)>, incorporated herein in its entirety by reference.

Because, as research suggests, the treatment of myocardial infarction is most effective with localized injections into affected surrounding tissue, a significant challenge in such treatment is to accurately characterize and localize affected surrounding tissue. General techniques for identification and localization of diseased or damaged myocardial tissue has generally involved pre-treatment invasive surgery, angiography by fluoroscopy, and/or electrocardiography. These techniques, however, are generally limited to providing an indistinct and/or equivocal diagnosis and may be expensive or have harmful side effects.

Certain techniques of tissue analysis have been developed to generally identify ischemic tissue and/or a tissue's metabolic state. Some of these techniques involve the use of optical spectroscopy and catheters combined with optical fiber probes. For example, such as for purposes of revascularization or other surgery, techniques have been developed for spectroscopically measuring oxygenation in myocardial tissue, as described in Kawasugi M., et al., “Near-infrared monitoring of myocardial oxygenation during ischemic preconditioning”, Ann Thorac Surg. 2000 June; 69(9): 1806-10), Nighswander-Rempel S P et al., “Regional variations in myocardial tissue oxygenation mapped by near-infrared spectroscopic imaging”, J Mol Cell Cardiol., 2002 September; 34(9): 1195-203, and Thorniley M S et al., “Application of near-infrared spectroscopy for the assessment of the oxygenation level of myoglobin and haemoglobin in cardiac muscle in-vivo”, Biochem Soc Trans. 1990 December; 18(6): 1195-6.), each incorporated herein in their entirety by reference. Other methods have been developed that are directed toward reviving merely stunned or hibernating tissue. These methods, such as those described in U.S. Pat. No. 5,865,738 by Morcos, et al., incorporated herein in its entirety by reference, generally depend on first injecting reagents into an area of interest in order to induce and subsequently detect metabolic activity.

None of these optical probe catheter technologies, however, have been developed toward combining the diagnosis of diseased tissue in a catheter together with its concurrent treatment, and in particular identifying and optimally localizing myocardial infarct and surrounding affected myocardial tissue in concurrence with its treatment.

### SUMMARY OF THE INVENTION

The system and methods of the present invention provide a safe, effective apparatus and method for in vivo characterization and concurrent treatment of tissue affected by myocardial infarction. The embodiments of the invention identify and locate infarcted tissue and the affected surrounding myocardial tissue for purposes of diagnosis (e.g. the state of viability) and subsequent treatment. The embodiments of the invention provide an integrated treatment system that operates in tandem with an identification system.

The inventive apparatus includes a catheterized optical probe connected to a spectroscopic analysis system programmed to identify (in vivo) and accurately locate infarcted myocardial tissue and various types of surrounding tissue affected by the infarction. The catheter further includes an integrated treatment system which, with information provided by the analysis system, can be accurately positioned to effectively treat the infarcted and affected surrounding areas such as, in an embodiment, by accurately localizing treatment delivery to affected areas surrounding necrotic tissue (e.g. periinfarct areas). In an aspect, the treatment system comprises a needle injection apparatus for injecting various compounds and/or therapeutic agents (e.g. stem cells, gene therapy, etc.) intended for aiding in the regeneration of necrotic tissue and/or revitalization of affected surrounding tissue.

In an aspect of the invention, an apparatus for probing and treating internal body organs is provided that includes a catheter having a fiber probe arrangement with one or more treatment lumens. The apparatus further includes an analysis and treatment control system connected to the catheter which is programmed to characterize and locate damaged tissue via the fiber probe arrangement and configured to treat damaged tissue through the one or more treatment lumens.

In an embodiment of the invention, the apparatus further comprises a spectrometer connected to said fiber probe arrangement.

In an embodiment of the invention, the apparatus further comprises a needle tip inserter.

In an embodiment of the invention, the needle tip inserter incorporates the probe ends of one or more fibers of the fiber probe arrangement and a dispersal port for the one or more treatment lumens. In an embodiment of the invention, the needle tip inserter is partially retractable within said catheter so as to ease the advancement of said catheter in a patient while permitting optical analysis.

In an embodiment of the invention, the analysis and treatment control system is programmed to analyze spectroscopic data, the analysis of the spectroscopic data including distinguishing the types and conditions of tissue within and surrounding a patient's heart. In an embodiment of the invention, the spectroscopic data is selected according to predetermined wavelength bands that distinguish levels of particles, gas, and/or liquid contained in the tissue. In an embodiment of the invention, distinguishing the types and conditions of tissue within and surrounding a patient's heart includes characterizing and locating tissues associated with myocardial infarct. In an embodiment of the invention, characterizing and locating tissues associated with myocardial infarct includes identifying an area for treatment of myocardial infarction by locating and targeting an affected area surrounding a region of necrotic tissue. In an embodiment of the invention, characterizing and locating the tissues associated with myocardial infarct includes detecting levels of at least one of fibrosis, calcification, or oxygen content. In an embodiment of the invention, the analysis of said spectroscopic data includes chemometric analysis of said spectroscopic data in relation to previously obtained and stored spectroscopic data. In an embodiment of the invention, the chemometric analysis involves at least one technique including Principle Component Analysis (PCA) with Mahalanobis Distance, PCA with K-nearest neighbor, PCA with Euclidean Distance, Partial Least Squares Discrimination Analysis, augmented Residuals, bootstrap error-adjusted single-sample technique, or Soft Independent Modeling of Class Analogy.

In an embodiment of the invention, the analysis and control system is configured to perform spectroscopic scans across wavelengths within the range of approximately 300 to 2500 nanometers.

In an embodiment of the invention, the analysis of the spectroscopic data includes estimating relative distances between a distal end of the fiber probe arrangement and tissue analyzed by the spectrometer. In an embodiment of the invention, estimating the relative distances includes comparing the magnitudes of spectroscopic absorbance peaks associated with tissue or blood with magnitudes similarly obtained from previously stored spectroscopic absorbance data. In an embodiment of the invention, the relative distances includes comparing the magnitudes of the spectroscopic absorbance peaks obtained at different predetermined positions of the catheter relative to the tissue or blood. In an embodiment of the invention, estimating the relative distances includes comparing spectroscopic absorbance peaks associated with collection fibers having terminating ends separated longitudinally from each other at a predetermined distance.

In an embodiment of the invention, the one or more treatment lumens includes a conduit for delivering a fluid solution to damaged tissue.

In an embodiment of the invention, the one or more treatment lumens includes a conduit for delivering therapeutic laser energy.

In an embodiment of the invention, the catheter further incorporates one or more sensors. In an embodiment of the invention, the one or more sensors includes at least one temperature gauge, pH meter, oxygenation meter, or water content meter.

In an embodiment of the invention, the catheter further includes a biopsy sampler.

In an embodiment of the invention, the distal end of the catheter includes a guidewire branching from the catheter apart from the needle tip.

In an embodiment of the invention, a catheter for probing and treating myocardial infarct is provided including a fiber probe arrangement, one or more treatment lumens, and a distal end having a needle injection inserter. The inserter is integrated with one or more fiber probe ends from one or more fibers of the fiber probe arrangement and is integrated with one or more delivery ports from the one or more treatment lumens.

In an embodiment of the invention, the catheter includes an angle control wire for adjusting the angle of the distal end of said catheter.

In an embodiment of the invention, the catheter includes a gripping element about the proximate portion of the catheter, the gripping element having one or more control elements for controlling aspects of positioning the catheter and/or for delivering treatment.

In an aspect of the invention, a method for treating body tissue is provided including the steps of inserting into a patient a catheter integrated with a fiber optic analysis probe and a treatment delivery conduit, characterizing and locating the body tissue to be treated with light delivered and collected through said fiber optic analysis probe, positioning the catheter to deliver treatment with information obtained through said fiber optic analysis probe, and delivering a treatment through the treatment delivery conduit.

In an embodiment of the invention, the body tissue to be treated is associated with myocardial infarct. In an embodiment of the invention, locating the body tissue associated with myocardial infarct to be treated includes locating and targeting an affected area surrounding a region of necrotic tissue for delivery of a treatment through the treatment delivery conduit.

In an embodiment of the invention, characterizing and locating the body tissue associated with myocardial infarct to be treated includes obtaining spectroscopic data from radiation delivered to and collected from the tissue to be treated via the fiber optic analysis probe and comparing the spectroscopic data with previously stored data characteristic of tissues within and around a patient's heart in order to identify the type of tissue being analyzed and to locate the position of the tissue being analyzed relative to the catheter.

In an embodiment of the invention, characterizing the tissue to be treated involves comparing levels of gases, fluids, and/or compounds within typical normal tissues as compared to gases, fluids, and/or compounds within tissues associated with myocardial infarct. In an embodiment of the invention, the gases, fluids, and/or compounds are selected from the group including collagen, calcium, oxygen, hemoglobin, and myoglobin.

In an embodiment of the invention, obtaining spectroscopic data includes at least one of the methods including diffuse-reflectance spectroscopy, fluorescence spectroscopy, Raman spectroscopy, scattering spectroscopy, optical coherence reflectometery, and optical coherence tomography.

In an embodiment of the invention, characterizing the tissue to be treated involves chemometric analysis selected from the group of techniques including Principle Component Analysis (PCA) with Mahalanobis Distance, PCA with K-nearest neighbor, PCA with Euclidean Distance, Partial Least Squares Discrimination Analysis, augmented Residuals, bootstrap error-adjusted single-sample technique, and Soft Independent Modeling of Class Analogy.

In an embodiment of the invention, the spectroscopic data is obtained from radiation spanning wavelengths between approximately 300 to 2500 nanometers.

In an embodiment of the invention, the spectroscopic data is selectively collected in sub-ranges of radiation spanning approximately 300 to 1375 nanometers, 1550 to 1850 nanometers, and 2100 to 2500 nanometers.

In an embodiment of the invention, the radiation that is delivered and collected through the fiber optic probe is restricted to selectively narrow spans of wavelengths associated with identifying said tissues. In an embodiment of the invention, radiation is delivered to tissue or blood within a narrow range including 380 nanometers and scanned across a narrow range including 320 nanometers in order to identify the presence of collagen.

In an embodiment of the invention, locating tissues in relation to the catheter includes pre-operative steps of analyzing and comparing the wavelengths and magnitudes of spectroscopic absorbance peaks associated with tissues and blood surrounding the tissues.

In an embodiment of the invention, the wavelengths and magnitudes of spectroscopic absorbance peaks associated with tissues and blood is compared with previously obtained and stored spectroscopic absorbance data associated with a catheter approaching similar tissues in a blood medium.

In an embodiment of the invention, the distal end of said catheter includes an inserter integrated with terminating ends of the fiber optic probe and delivery conduit, the inserter suitably sharp for perforating targeted tissue.

In an embodiment of the invention, during the positioning of the catheter for delivery of treatment, the integrated inserter remains at least partially retracted in the catheter prior to perforation into tissue targeted for treatment and the fiber optic probe is functional while the inserter is at least partially retracted. In an embodiment of the invention, final positioning of the catheter for delivery of treatment includes extending the inserter out from the distal end of the catheter into the targeted tissue.

In an embodiment of the invention, prior to and during extension of the inserter, a wall of myocardial tissue before which the inserter is positioned is concurrently analyzed and monitored to prevent complete perforation of the inserter through the entire wall of myocardial tissue.

In an embodiment of the invention, the prevention of complete perforation includes monitoring the contents of tissue for a layer of pericardial fat positioned beyond the wall of myocardial tissue.

In an embodiment of the invention, delivering treatment through the treatment delivery conduit includes the injection of therapeutic agents. In an embodiment of the invention, the therapeutic agents include at least one of chemical agents, gene therapy agents, stem cell therapy agents, and/or cytotherapy agents.

In an embodiment of the invention, the therapy agents are chosen and delivered based on data collected during characterizing and locating the body tissue to be treated.

In an embodiment of the invention, the release of agents is monitored with the fiber optic probe and controlled using feedback from said monitoring.

In an embodiment of the invention, delivering treatment through the treatment delivery conduit comprises delivering therapeutic laser energy. In an embodiment of the invention, delivering therapeutic laser energy comprises canalizing infarct tissue for purposes of revascularization.

In an embodiment of the invention, the catheter is introduced into the patient in accordance with a percutaneous transluminal angioplasty.

In an embodiment of the invention, the catheter is introduced into the patient in accordance with percutaneous endoventricular delivery.

### BRIEF DESCRIPTION OF THE DRAWINGS

The structure, operation, and methodology of embodiments of the invention, together with other objects and advantages thereof, may best be understood by reading the following detailed description in connection with the drawings in which each part has an assigned numeral or label that identifies it wherever it appears in the various drawings. The drawings are not necessarily to scale, emphasis instead being placed upon illustrating the principles of the embodiments of the invention.

FIG. 1 is a schematic block diagram of an apparatus illustrating the general flow of system control, including identifying, localizing, and treating diseased internal tissues, in accordance with an embodiment of the invention.

FIG. 2A is an illustrative schematic diagram of the end of a catheterized optical probe and needle injection system that analyze myocardial tissue, in accordance with an embodiment of the invention.

FIG. 2B is an illustrative schematic side-profile view of the needle tip inserter portion of the probe of FIG. 2A.

FIG. 3 is a side-profile view of a distal end of a catheter having a control cable, in accordance with an embodiment of the invention.

FIG. 4 is an illustrative view of a handle assembly, in accordance with an embodiment of the invention.

FIGS. 5A-5D are illustrative views showing the sequential steps of performing an optical-probe guided injection treatment procedure for infarcted myocardial tissue, in accordance with an embodiment of the invention.

FIGS. 6A-6F are illustrative views showing various embodiments of fiber probe tip arrangements according to embodiments of the invention.

FIG. 7A is an illustrative perspective view of a catheter having a guidewire sheath according to an embodiment of the invention.

FIG. 7B is an illustrative cross-sectional view of the distal end of the catheter of FIG. 7A.

FIG. 7C is a schematic diagram of the distal end of the catheter of FIGS. 7A-7B approaching a region of interest via a vessel of a heart.

FIG. 8 is a chart of an absorbance spectrum taken across a range of wavelengths comparing various body tissues and fluids.

FIG. 9 is a chart of an absorbance spectrum taken across a range of wavelengths comparing various types of myocardial tissue associated with normal and damaged tissue states.

FIG. 10 is a chart of absorbance spectra for two different fiber probe configurations at various positions relative to adjacent layers of myocardium and fat tissue.

### DETAILED DESCRIPTION OF EMBODIMENTS

In one aspect of the invention, an apparatus and method are provided for treating tissue associated with myocardial infarction by integrating an inspection system for locating tissue to be treated with a treatment delivery system.

The preferred embodiments of the invention employ spectroscopic analysis with any two or more single wavelengths or one or more narrow wavelength bands, or a whole wavelength range to identify and localize myocardial infarct lesions in vivo. The light signal scattered or emitted from an illuminated area provides information about a change in tissue chemical components (such as water content, oxygenation, pH value, collagen, proteoglycans, calcium), tissue structures (such as cell size, types), inflammatory cellular components (such as T lymphocytes, macrophages, and other while blood cells), that help characterize states of tissue edema, tissue necrosis, tissue fibrosis, and/or tissue calcification or other conditions which typically result from myocardial infarct (“MI”).

The ability to identify myocardial infarcts is dependent upon the time that has elapsed since the ischemic event took place. Infarcts resulting in sudden cardiac death and are less than 12 hours old are usually not apparent upon gross examination. The infarcted tissue may become edematous and inflamed. Changes during this time period are histochemical and require adjunctive staining to identify the affected area of necrosis. After 24 hours, however, pallor is often grossly present due to stagnated blood within the lesion. Acute inflammation occurs within the first several days, followed by granulation over a couple of weeks. Eventually, the tissue becomes more fibrous and less vascularized. Some long resident infarcted tissue may become calcified.

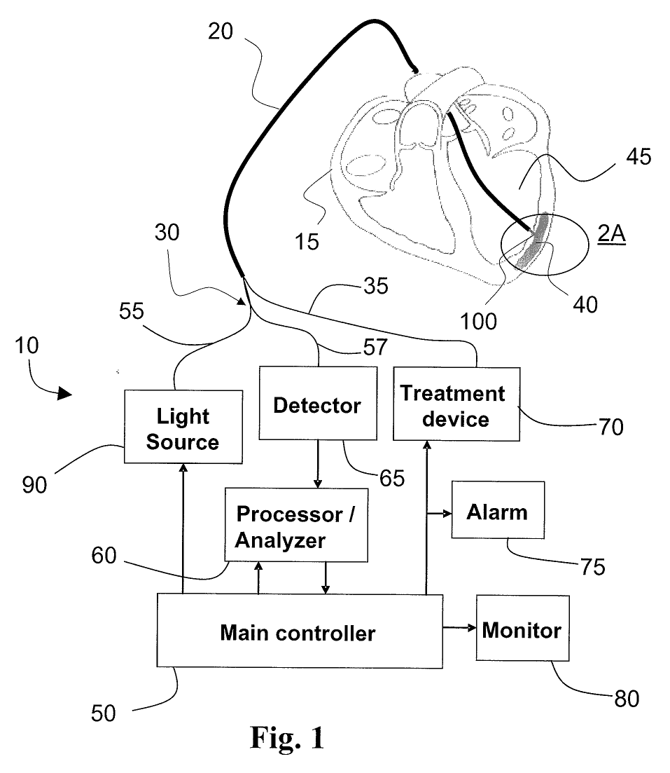

FIG. 1 is a schematic block diagram of an apparatus illustrating the general flow of system control, including identifying, localizing, and treating diseased internal tissues, in accordance with an embodiment of the invention. Referring to FIG. 1, the block diagram 10 shows fiber cables 30 and a treatment delivery conduit 35 extending through a probe/treatment catheter 20, which is inserted into a heart 15. Arrows between the boxed elements of diagram 10 indicate the general flow of system control, originating from main controller 50, which includes a programmed processor and data storage elements (not shown) for routing commands and data to and from various other system components.

Main controller 50 is connected to a light source 90 which delivers radiation through optical delivery fibers 55 to illuminate target tissue 40 of the heart 15. As described in further detail below, light source 90 is preferably of the type that can selectively produce light in one or more wavelengths within the visible and/or near-infrared spectrum, including single LED varieties. Main controller 50 operates a processor/analyzer 60 that is connected to a detector 65, which is connected to collection fibers 57 that extend to the distal end 100 of catheter 20. The detector 65 converts optical signals to electrical/digital signals. The detector 65 and processor/analyzer 60 are also preferably of the type for processing near infrared radiation. Numerous commercially available spectrometers capable of analyzing visible radiation and also near-infrared radiation in accordance with embodiments of the invention such as, for example, an IntegraSpec™ NIR Microspectrometer from Axsun Technologies, Inc.

Referring to FIG. 1, also connected to the main controller 50 is a treatment device 70 which supplies a treatment delivery conduit 35 with selected treatment agents as described in further detail below. An alarm 75 is interconnected with the controller 50 and treatment device in the event the system detects a problem and treatment operations should be suspended (e.g. accidental penetration into non-myocardial tissue). In an embodiment, a monitor 80 and various input devices, for example, a keyboard, mouse, etc. (not shown), can provide an operator with feedback, status information, and control.

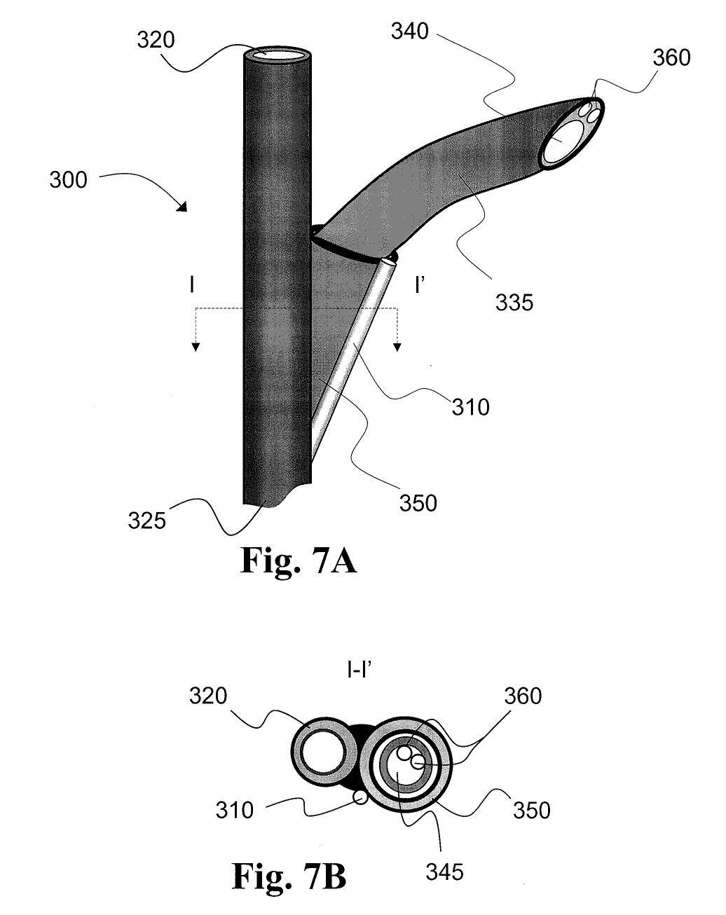

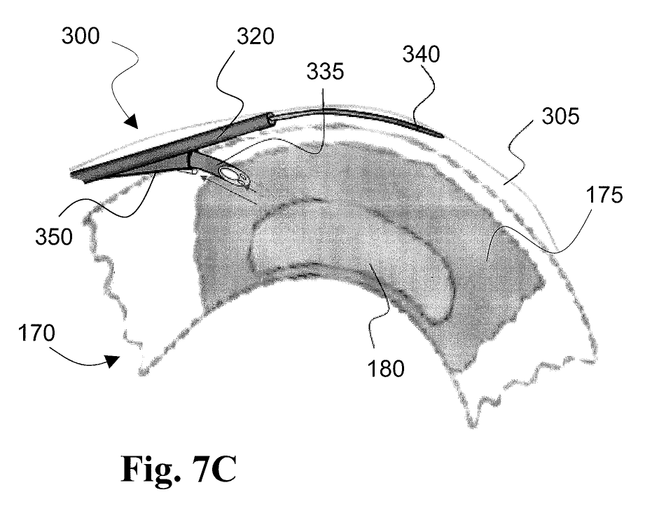

In an embodiment of the invention, the catheter 20 is introduced into a human body and approaches the affected tissue via vessels and cavities through which the catheter may slide through. In an embodiment of the invention, a guide catheter (not shown) may be operated in a manner consistent with percutaneous endoventricular delivery. For example, the guide catheter enters the body via a peripheral artery, such as femoral artery, then into the aorta, and then into the left (atrium and ventricle) heart cavity. Alternatively, the guide catheter is inserted into the body via a peripheral vein, such as basilic or femoral vein, then into the vein cave, and then into the right heart (atrium and ventricle) cavity. Other embodiments, such as those described below in reference to FIGS. 7A-7C, allow for a method of approaching affected tissue via adjacent heart vessels.

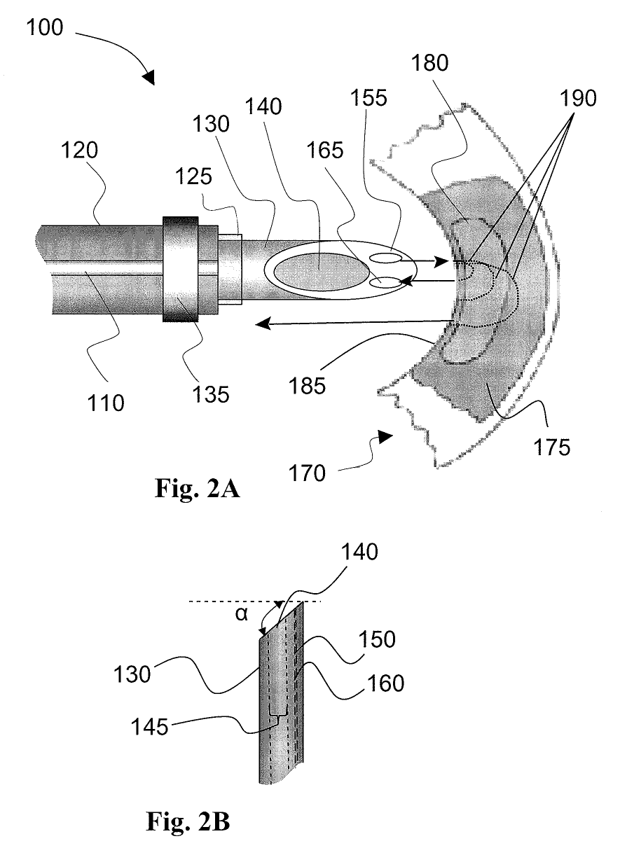

Referring to FIG. 1, the distal end 100 of catheter 20 is shown within a heart cavity 15 penetrating a targeted myocardial infarct region 40 in the cavity 45 wall. The processor and analyzer 60 provide controller 50 with spectral absorbance feedback as the catheter 20 is positioned in the cavity 45 and into its inner walls. With appropriate chemometric data, controller 50 is pre-programmed to identify infarcted tissue and surrounding affected tissue in relation to the distal portion 100. With use of the tissue identification results (e.g. magnitudes of spectroscopic absorbance peaks taken at various positions of the catheter), controller 50 is programmed to accurately determine the optimal position of the treatment component (shown below in FIG. 2A) of catheter 20 and amount of treatment agent to be discharged. Positioning may be performed in varying degrees of programmed interactivity with an operator (not shown). For example, data from the probe could be processed and displayed to show general indications of tissue conditions and/or position. Alternatively, a real-time spectral readout could be continuously displayed for the operator to judge independently.

FIG. 2A is an illustrative schematic diagram of the end of a catheterized optical probe and needle injection system that analyze myocardial tissue, in accordance with an embodiment of the invention. FIG. 2B is an illustrative schematic side-profile view of the needle tip inserter portion of the probe of FIG. 2A. Referring to FIG. 2A, an embodiment of the distal portion 100 of a catheter is shown in accordance with embodiments of the inventive apparatus and methods. A protective outer sheath 120 surrounds a catheter body 125. The end of catheter body 125 is integrated with an inserter 130. The body of the catheter may be a flexible tube, which may be bifurcated at the injection lumen, or treatment lumen, or just an empty pathway to allow for the inclusion of one or multiple optical fibers while maintaining the fluid path for a treatment solution or as a transfer path for a treatment device. The catheter body is allowed to be partially pulled back, or retracted, inside the catheter sheath 120 while the catheter enters into the human body. The catheter sheath 120 also allows the catheter body 125 to move partially forward in order to push the suitably sharp inserter 130 outside of the catheter sheath 120 and to puncture the target myocardial tissue 170 for at least one of a diagnosis and a treatment procedure.

Inserter 130 preferably comprises stainless steel or similar material suitable for perforating myocardial tissue by moderate forward pressure. Housed within the inserter 130 is a fiber probe arrangement comprising one or more delivery fibers 150 and collection fibers 160 with, respectively, fiber ends 155 and 165, also referred to as terminating ends, being connected at their opposite ends to corresponding sources and/or detector/analyzer(s). The terminating ends 155 and 165 are fixed within inserter 130, for example, using an epoxy adhesive or metal solder. The fiber ends 155 and 165 are polished such that they have oblique angles with respect the external surface of inserter 130. Inserter 130 also includes a treatment port 140 or dispersal port for one or more treatment lumens, for delivery of treatment to the area surrounding and including a region of infarcted myocardial tissue 180. Treatment port 140 is connected through a treatment supply conduit 145 which can be connected to a treatment device as described in reference to FIG. 1.

Inserter 130 is sized preferably at about 18 to 27 gauge with a length from about 3 to 30 mm depending on the particular application (i.e. the density of tissue material, the preferable depth of penetration, etc.). In an embodiment of the invention, the angle α relative to a perpendicular of the terminating end of the inserter has a range of approximately 25 to 75 degrees (see FIG. 2B), sufficient to protect the terminating ends of optical and treatment components, for example, terminating ends 155 and 165, while promoting easier penetration into tissue.

In accordance with operation of embodiments of the invention, the catheter's distal portion approaches a cross-section of myocardial tissue area 170 of an inner heart cavity's wall which includes regions of myocardial infarcted tissue 180 and affected surrounding tissue 175. Source radiation paths represented by lines 190 emanate from delivery fiber end 155 into the heart cavity's interior wall edge and from there penetrate and interact with surrounding myocardial tissue. Return radiation emerges out of the wall of myocardial tissue area 170 and is collected by collection fiber ends 165 and that of fiber 110, then delivered to a detector/analyzer (as shown in FIG. 1).

The amount of detectable signal and the depth of the path of the collected signal is generally proportional to the degree of latitudinal separation between delivery and collection fibers. While having signal power levels sufficiently low not to damage targeted tissue, a separation of less than 1.5 mm is preferable for receiving an adequate collection signal. In another embodiment, in order to receive signals from varying depths of blood and tissue concurrently, one or more additional optical fibers, such as collection fiber 110, can be integrated with the outside area of protective outer sheath 120. Fiber 110 is can be fixed to sheath 120 with a ring 135 or by other various means of attachment known to those of ordinary skill in the art. In an embodiment, an inside collection fiber end 165 can be separated from a signal fiber end 155 by approximately 1.5 mm and collection fiber end 110 can be separated from signal fiber end 150 by approximately 1.0 mm.

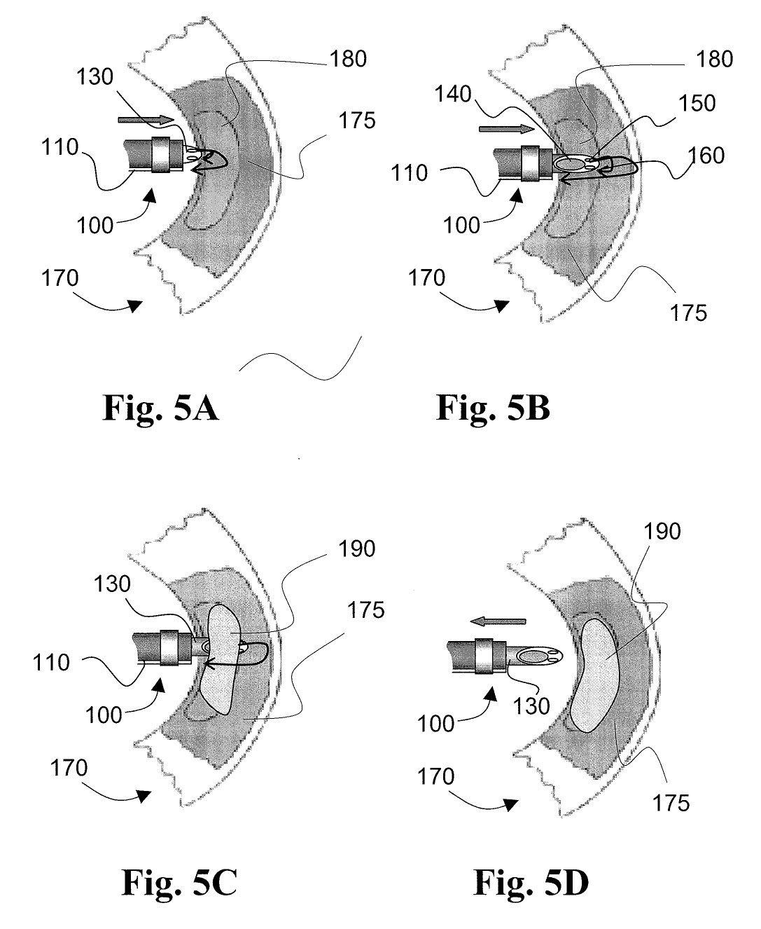

In an embodiment, during the analysis and treatment procedures, at least one collection fiber 110 can remain outside of the heart wall tissue 15, unlike fiber ends 155 and 165. Additional details on this embodiment are described below in reference to FIGS. 5A-5D. This approach provides additional collection of optical signals relative to the heart wall surface, while fibers 150 and 160 are embedded in the heart wall tissue. With information known about the relative positions between the collection fiber ends and data collected from each end, the depth of penetration of the catheter into the targeted tissue can be reasonably calculated.

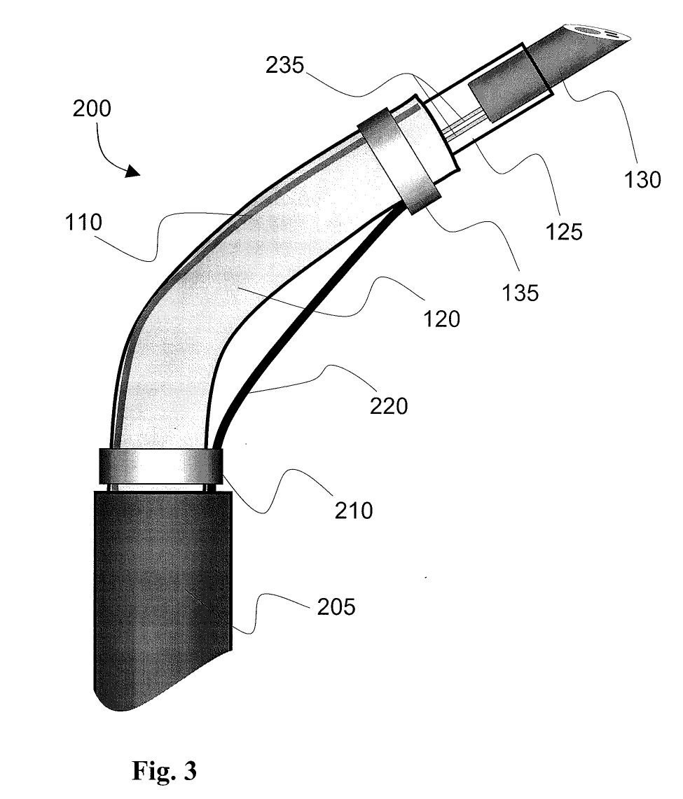

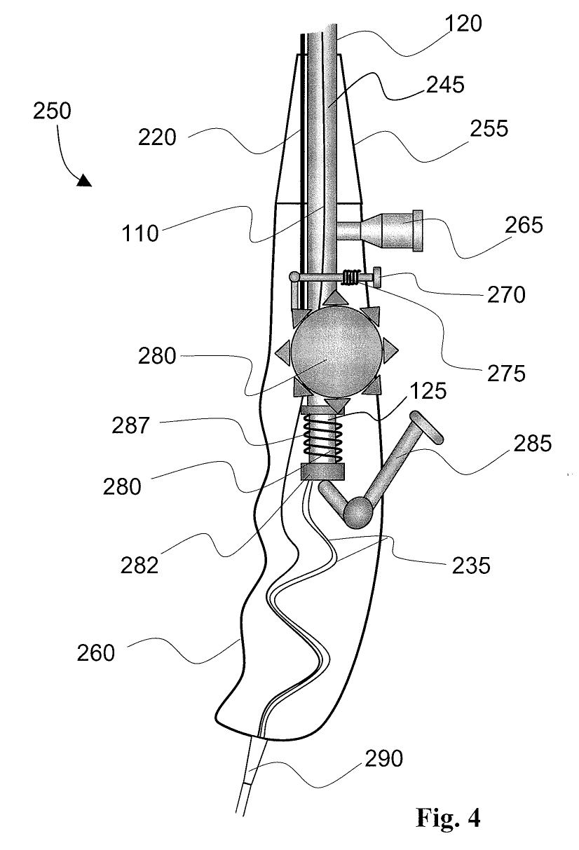

FIG. 3 is a side-profile view of a distal end of a catheter having a control cable, in accordance with an embodiment of the invention. Referring to FIG. 3, a distal end of the catheter 200 includes a control cable 220 for manipulating its angle as it emanates from a protective outer catheter sheath 205. A ring 210 has holes (not shown) through which cable 220 and fiber line 110 may slide through. Ring 210 is also slidable along catheter sheath 120. Ring 135 is fixed to catheter sheath 120 and holds the ends of fiber line 110 and cable 220 in place. After distal end 200 is extended through catheter sheath 205, cable 220 can then be retracted, for example, via a control knob, such as the control knob 280 shown in FIG. 4, to bend the distal portion 200 at a desired angle, providing additional control of the catheter. Various non-toxic lubricants and compounds for resisting a build-up of blood on the surfaces of the catheter may be applied prior to an operation. Fibers 235 extend through a catheter body 125 with integrated inserter 130 as in previously described embodiments.

In an embodiment of the invention, one procedure for approaching a target myocardial area applying the embodiment at FIG. 3 is in accordance with percutaneous endoventricular delivery. For example, catheter 200 is introduced into a body via a peripheral artery, such as a femoral artery, and in through a ventricle of the heart, and then toward an area of interest where inserter 130 can emerge.

FIG. 4 is an illustrative view of a handle assembly, in accordance with an embodiment of the invention. Referring to FIG. 4, a handle assembly 250 provides a way for an operator to manually control movement (e.g. pulling, pushing, turning) and other operations of a catheter in accordance with embodiments of the invention. Catheter sheath 120, control cable 220 and fiber line 110 enter handle assembly 250 through an upper handle segment 255 and then into lower handle segment 260. A flush port 265 allows a treatment agent to enter sheath 120. Sheath 120 can operate as a treatment delivery conduit for subsequent passage and delivery of a treatment agent to a patient (e.g. out through treatment port 140 as shown in FIGS. 2A-2B). A control knob 280 retracts and extends control cable 220 to adjust the angle of the distal end of the catheter 200, as shown in FIG. 3. A release button 270 releases tension on control wire 220. In an embodiment, the button 270 is spring loaded (in a non-release position) by a spring 275. A lever 285 can apply force to head 282 to actuate movement of catheter body 125 and an inserter tip (e.g., inserter 130 shown in FIGS. 2-3) into a target tissue area. Catheter body 125 is spring loaded by spring 287 which holds inserter 130 in a normally retracted position. Fibers 155 and 110 extend through lower handle segment 260 and out through a conduit 290 to corresponding sources or detectors (e.g., source 90 and detector 65 as shown in FIG. 1). In an embodiment, fiber 110 is a collection fiber and fibers 135 include collection and delivery fibers.

FIGS. 5A-5D are illustrative views showing the sequential steps of performing an optical-probe guided injection treatment procedure for infarcted myocardial tissue, in accordance with an embodiment of the invention. Referring to FIGS. 5A-5D, a catheter's distal end 100 and inserter 130 is shown in various positions during an analysis and treatment procedure in accordance with embodiments of the invention. Referring to FIG. 5A, inserter 130 is partially retracted within distal end 100 as it approaches the inside surface of a heart wall 170. The needle tip inserter 130 is partially retracted within said catheter so as to ease the advancement of said catheter in a patient while inserter 130 is sufficiently extracted so that the optical probe remains functional, permitting optical analysis to occur through inserter 130. Prior to and during extension of said inserter, the wall 170 of myocardial tissue before which the inserter 130 is positioned can be concurrently analyzed and monitored to prevent complete perforation of said inserter through the entire wall 170 of said myocardial tissue. The optical analysis system operates and examines inside surface and interior of heart wall 170 during the approach, determining the catheter's distance from surface and diagnosing the condition of myocardial tissue therein. In order to prevent a complete perforation of the inserter 130 through the wall of myocardial tissue, the contents of tissue for a layer of pericardial fat positioned beyond the wall 170 of myocardial tissue can be monitored. Upon diagnosing and locating myocardial infarct region 180 and affected surrounding tissue 175, distal end 100 is optimally positioned for delivering treatment to the region.

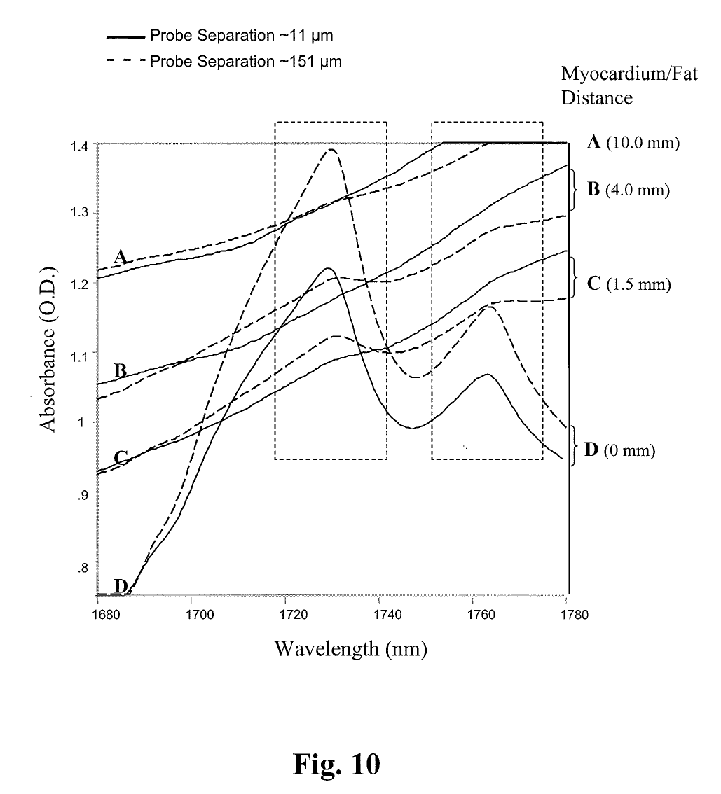

Referring to FIG. 5B, after distal end 100 is positioned for treatment, inserter 130 is driven out through the catheter body and into the adjacent region of myocardial tissue, exposing treatment port 140 within the wall 170 of myocardial tissue. While the probe end of collection fiber 160 becomes embedded into myocardial tissue, the intensity and spectral features of the optical signal collected by fiber 110 (while not embedded) can be compared to that collected by fiber 160 to better assess the puncture position of inserter 130. Being positioned externally to the heart tissue, collection fiber 110 will likely receive a stronger return signal from delivery fiber 150 in order to better assess proximity with and avoid a perforation of the outer heart wall surface, which could be highly damaging or fatal. A simulative set of signals in accordance with the operation of this feature is described below in reference to FIG. 10.

Referring to FIG. 5C, treatment port 140 then injects treatment agent 190 into the affected areas.

Referring to FIG. 5D, the distal end of the catheter 100 is withdrawn from the area.

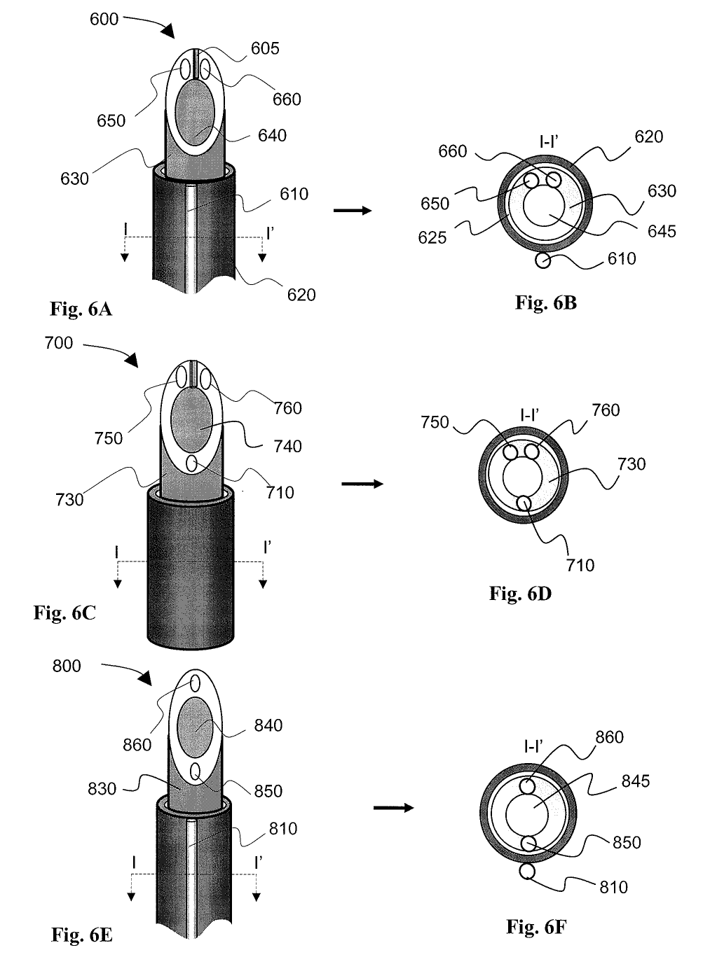

In an embodiment, a tube or passageway inside of the catheter (e.g. the interior of catheter body 125 shown in FIGS. 2-4) can be used as a conduit to transfer the treatment fluid such as, for example, stem cell suspension or drug solution, into the target tissue for cytotherapy, gene therapy and/or chemical therapy in a narrow local area inside the heart wall. The optical probe system can monitor the spread of therapeutic agents in tissue while they are delivered. A controller (e.g. controller 50 of FIG. 1) can be programmed and configured to identify spectra associated with the particular treatment agent administered, and thus enable identification and tracking of the agent during and after dispersal. The catheter may also provide a conduit through which other treatment tools can deliver treatment to the affected area, e.g. additional treatment lumens or a treatment fiber with high power laser energy to canalize infarct tissue for revascularization as described by Lauer B., et al., “Catheter-based percutaneous myocardial laser revascularization in patients with end-stage coronary artery disease.” J Am Coll Cardiol. 1999 Nov. 15; 34(6):1663-70, incorporated herein in its entirety by reference. For example, in embodiments of the invention, one or more of fibers 150 or 160 of FIG. 2 or fiber 710 of FIGS. 6C-6D could be adapted and used to deliver therapeutic laser energy. These fibers could be, for example, switched between use for delivery/collection for purposes of analysis and use for delivering therapeutic laser energy.

In embodiments of the invention, alternate fiber optic probe arrangements are provided. FIG. 6A shows an illustrative perspective view of an alternate probe tip arrangement 600, including a light blocking divider 605 between the terminating ends of a delivery fiber 650 and collection fiber 660. FIG. 6B shows a cross-sectional illustrative view of the probe tip arrangement of FIG. 6A. Fibers 650 and 660 extend through a catheter sheath 620 and catheter body 625, to an inserter 630 having a treatment delivery port 640 that provides an output to a treatment delivery conduit 645. A collection fiber 610 extends and terminates along sheath 620 at a position longitudinally separated from the terminating ends of fiber 650 and 660. Light-blocking divider 605 can help minimize the amount of signal directly traveling to (or leaked between) delivery fiber 650 and collection fiber 660 prior to traveling through a targeted tissue area.

FIG. 6C shows an illustrative perspective view of an alternate probe tip arrangement 700, including a collection fiber 710 having a terminating end integrated in an inserter 730. FIG. 6D shows a cross-sectional illustrative view of the probe tip arrangement of FIG. 6C. The probe end of collection fiber 710 is longitudinally separated from fibers 750 and 760 as in previously described embodiments, however, its probe end will remain longitudinally fixed with respect to the ends of fibers 750 and 760 when inserter 730 emanates from a sheath 720 and retracts. Fixing the separation between the probe ends of fibers 750, 760, and 710 can thus reduce the level of analysis required during movement of inserter 730 and increases the overall proximity to and reception of signals associated with treatment agents delivered from a treatment delivery port 740, thus providing enhanced analysis of the quantity, movement, and progress of delivered treatment agents. In addition, being fixed within sheath 720, fiber 710 can remain less exposed to external components (e.g. blood and tissue), thus reducing the likelihood of damage to external tissue and fiber 710.

FIG. 6E shows an illustrative perspective view of an alternate probe tip arrangement 800, including the three longitudinally separated fibers 850, 860, and 810. FIG. 6F shows a cross-sectional illustrative view of the probe tip arrangement of FIG. 6E. The probe ends of fibers 850 and 860 are separated along an inserter 830 at opposing longitudinal ends of a treatment delivery port 840 that provides an output to a treatment delivery conduit 845. Longitudinally separating the probe ends of fibers 850 and 860 can reduce the level of signal leaking between the fibers and also increases the overall reception of signals associated with treatment agents delivered from a treatment delivery port 740, thus providing enhanced analysis of the quantity, movement, and progress of delivered treatment agents.

In other embodiments, the inventive catheter incorporates a biological, electric, or chemistry-based sensor or tool connected with a metal fiber, or other structural or reinforcing wire elements permitting additional diagnosis or monitoring of target tissue, e.g. tissue temperature, pH, oxygenation, water content, other chemical composition and/or even tissue biopsy via the catheter body. In an embodiment, the catheter includes one or more sensors. The sensors can be at least one of a temperature gauge, pH meter, oxygenation meter, and water content meter. In another embodiment, the catheter includes a biopsy sampler. In an embodiment, a sensor wire can travel along a similar path as that of fibers 150 or 160 shown in FIG. 2 and a sensor/transducer could be situated in, for example, needle tip inserter 130 shown in FIG. 2. In an embodiment, a biopsy can be performed by extracting tissue or other materials through treatment port 140 and suctioning them to the proximate end of the catheter. A cutting device (not shown) could be incorporated into needle tip inserter 130 and treatment port 140 in order to detach tissue for extraction.

FIG. 7A is an illustrative perspective view of a catheter 300 having a guidewire sheath 320 according to another embodiment of the invention. FIG. 7B is an illustrative cross-sectional view of the distal end of the catheter of FIG. 7A. FIG. 7C is a schematic diagram of the distal end of the catheter of FIGS. 7A-7B approaching a region of interest via a vessel of a heart. Guidewire sheath 320 and a probe and treatment end 350 bifurcate from a protective catheter sheath 325. Probe and treatment end 350 includes an angled inserter 335 through which a treatment delivery conduit 345 transfers a treatment agent out to a treatment port 340. Fibers 360 also extend through the treatment delivery conduit 345 and to the probe and treatment end 350, terminating at the end of inserter 335. Inserter 335 remains partially retracted while the catheter is fed through the patient in its approach to myocardial wall 170, infarcted area 180, and affected surrounding area 175 while the optical probe components can continue to function. As in previously described embodiments, inserter 335 can then extend from the probe and treatment end 350 into adjacent myocardium. The angle of divergence between guide wire sheath 320 and inserter 335 is preferably between 15 and 90 degrees, sufficient to allow puncturing of adjacent myocardial tissue. This embodiment enables the catheter to approach the myocardium wall 170 substantially through blood vessels such as blood vessel 305.

One procedure for approaching target myocardial areas applying the embodiment illustrated at FIGS. 7A-7C is similar to that of percutaneous transcoronary angioplasty (PTCA). For example, guide wire 340 is introduced into a body via a peripheral artery, such as femoral artery, into the aorta, then into the coronary artery system through the coronary ostium at the beginning of the aorta arch. Alternatively, it is introduced into body via a peripheral vein, such as basilic or femoral vein, then into the coronary vein system through an opening in the right atrium. The catheter is then finally advanced into a coronary blood vessel (artery or vein) lumen 305 to the area of interest 175, where inserter 335 can emerge and perforate the vessel's walls in order to perform additional analysis and to apply treatment.

### Spectroscopic Tissue Analysis and Diagnosis

A number of techniques with the use of embodiments of the invention, including spectroscopy, can be employed for diagnosing tissue conditions, including myoacardial infarct. Spectroscopic analysis techniques used alone or in combination include, but are not limited to, fluorescence spectroscopy, visible spectroscopy, diffuse-reflectance spectroscopy, infrared or near-infrared spectroscopy, scattering spectroscopy, optical coherence reflectometery, optical coherence tomography, and Raman spectroscopy.

To optimize speed, it is preferable that, during operation, the source of radiation be limited and selectable in particular wavelength band ranges known to provide optimal feedback about the types of tissue being targeted (e.g. myocardial infarct and surrounding tissues and blood). A variety of light sources can be used to provide radiation in this manner, such as one or multiple lasers, one or multiple LEDs, a tunable laser with one or multiple different wavelength ranges, Raman amplifier lasers, and a high-intensity arc lamps. These light sources can provide the desired optical radiation region by sequential tunable scanning or by simultaneously spanning the desired wavelength band(s). Wavelength tuning during scans should preferably occur between about a couple of microseconds to less than one second in order to avoid motion related artifacts (e.g. those associated with a pulsing heart).

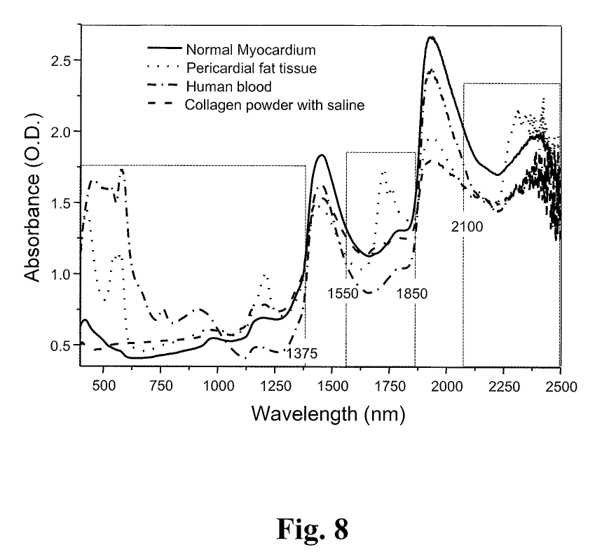

FIG. 8 is a chart of a sample absorbance spectrum taken across a range of wavelengths comparing various types of bodily tissues and fluids including normal myocardium, fat tissue, blood, and collagen. Such spectra and the peaks associated with the various types of tissue and fluids can be used as a basis for performing the identification techniques described herein according to embodiments of the invention. Peak regions associated with collagen, for example, that are not generally present or associated with normal myocardium, blood, or fat tissue can be detected and analyzed to distinguish and characterize a fibrous region adjacent an infarct region.

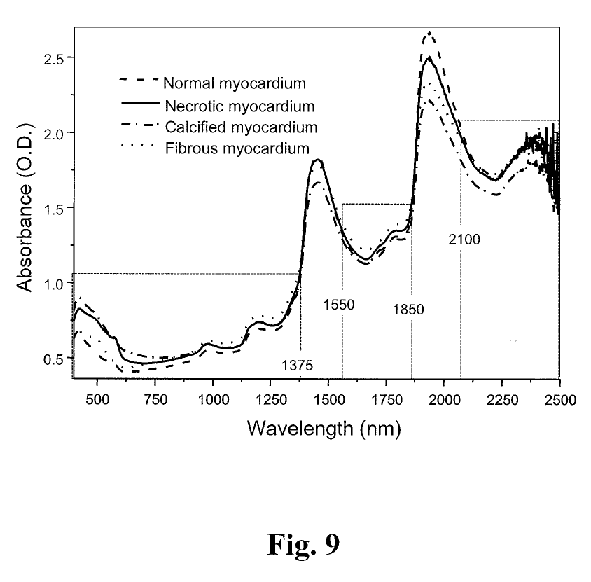

FIG. 9 is another chart of a sample absorbance spectrum taken across a range of wavelengths comparing various types of bodily tissues and fluids including normal myocardium, calcified tissue, fibrous tissue, and necrotic tissue. Peak regions associated with necrotic tissue, for example, that are not generally present or directly associated with normal myocardium, can be detected and analyzed to distinguish, characterize, and locate an infarct region. Peak regions associated with calcified and fibrous tissue, for example, can be used to help identify and locate surrounding tissue affected by an infarct.

In embodiments of the invention, data from multiple similar spectra scans across varying wavelength ranges with known varying backgrounds in multiple living or deceased subjects can be compiled and analyzed to develop a model to be programmed in coordination with optical, processor/analyzer, and controller components of embodiments of the invention described herein (e.g. those components of FIG. 1).

In an embodiment of the invention, a detector and processor/analyzer (such as, for example, the detector 65 and processor/analyzer 60 of FIG. 1) perform spectroscopic scans across wavelengths having a range of approximately 300-2500 nm. In an embodiment, the spectroscopic absorbance data is collected across sub-ranges of radiation spanning approximately 300-1375 nm., 1550-1850 nm., and 2100-2500 nm. In an embodiment, radiation is delivered to tissue or blood at a narrow range including 380 nanometers and scanned across a narrow range including 320 nanometers in order to identify the presence of collagen.

Additional optical elements may be integrated into the delivery and collection systems in order to improve the quality of and/or provide additional control over signals. For instance, filters of various types (e.g. longpass, lowpass, bandpass, polarizing, beam splitting, tunable wavelength, etc.) could be placed in between the light source and delivery fibers or between the detector and collection fibers depending on application parameters. For example, a coating of appropriate polymer on the ends of fibers could serve as a filter.

A number of different types of detectors may be suitable for initial collection and signal processing of radiation received through collection fibers. A detection device may include one or more (individual or arrayed) detector elements at the proximal portion of collection fiber(s) in accordance with embodiments of the invention, such as InGaAs, Silicon, Ge, GaAs, and/or lead sulfide detectors for detecting optical radiation emitted from illuminated tissue.

The detector converts the collected optical signal into an electrical signal, which can be subsequently processed into spectral absorbance or other data using various known signal processing techniques. The electrical signal is preferably converted to digital spectral data for further processing using one or more discrimination algorithms. Using collected spectral data, discrimination algorithms may execute morphemetry measurements, chemical analysis, or perform similar calculations and correlate the results with pre-stored model data to provide a diagnosis of targeted tissue. Model data representing the relationship between spectral data and tissue characteristics is preferably developed from the analysis of large amounts of patient in vivo data or ex vivo data simulating in vivo conditions. The models can be developed with chemometric techniques such as Principle Component Analysis (PCA) with Mahalanobis Distance, PCA with K-nearest neighbor, PCA with Euclidean Distance, Partial Least Squares Discrimination Analysis (PLS-DA), augmented Residuals (PCA/MDR), and others such as the bootstrap error-adjusted single-sample technique (BEST), and Soft Independent Modeling of Class Analogy (SIMCA).

For aiding in a careful approach and interrogation (e.g. preventing perforation of a myocardial wall into an outside fat layer) by the inventive probe, absorbance peaks for distinguishing the myocardium, fat, blood, collagen and/or fibrin are discernable with use of the above described algorithmic techniques. Several high-speed commercially available near infrared spectrometers are available for obtaining the desired spectral readings including the IntegraSpec™ NIR Microspectrometer from Axsun Technologies, Inc., the Antaris FT-NIR spectrometer, and a FOSS NIR System, model 6500. The models were selected for their high speed and performance in the spectral regions of interest (i.e. near infrared). A number of other comparable high-speed spectrometers would also be suitable. Limiting scanning to generally flat, narrow regions of spectroscopic bands (e.g. 1550 to 1800 nanometers) is preferable for purposes of speed while maintaining reasonable accuracy. In an embodiment, spectroscopic scans are performed across wavelengths having a range of approximately 300-2500 nm. While probing for particular tissue/fluid types or conditions, it may be preferable to employ such techniques as tissue fluorescence spectroscopy and/or selectively focus transmission bands to excite specific scanning ranges. For example, a radiation excitation peak for collagen at approximately 380 nm occurs when radiation of approximately 340 nm is delivered.

In order to accurately position the catheter for providing treatment, spectroscopic analysis can also distinguish the types and conditions of tissue within and surrounding a heart, including three major diseased states associated with myocardial infarct: necrotic tissue, calcified tissue, and fibrous tissue. The chosen discrimination algorithm can compare collected data with pre-programmed spectra data of myocardial tissue to categorize both the condition and relative location (to the catheter tip) of a tissue area. Based on spectral analysis, the tissue can be characterized as being normal myocardial tissue, affected tissue surrounding a myocardial infarct region (edema inflammatory zone), fibrosis, and/or necrotic or calcified myocardial infarct lesions. Spectral analysis reflecting high degrees of endema content and/or inflammation indicate a region of tissue surrounding infarcted or necrotic tissue.

The intensity of peaks associated with various tissue types can generally be correlated with the distance the probe is from the targeted tissue and from data related to the medium in which the probe is in (e.g. blood, myocardium, fat). Thus, analysis of spectroscopic absorbance data can include estimating relative distances between a distal end of a fiber probe arrangement and tissue to be analyzed. For instance, in preparing and programming an embodiment of the invention for operation, experiments can be performed on various in vivo or ex vivo samples, including samples having measured thicknesses of layers of myocardium and surrounding fat tissue. Fat tissue surrounding the heart is known to generate absorbance peaks, for example, at approximately 1728 and 1766 nanometers. Data can be collected on the changes (e.g. intensity) in these peaks as the needle tip of an embodiment approaches fat tissue through a layer of myocardium. Collected data would correlate, for example, peak intensity with the otherwise measured distances between the needle tip and the fat layer.

FIG. 10, for example, is a chart of absorbance spectra for two different fiber probe configurations at various positions relative to adjacent layers of myocardium and fat tissue. Absorbance spectra were measured through two probe configurations, one having a relatively small source-detector separation (approx. 11 μm) and another having a relatively large separation (approx. 151 μm), designated by solid and dashed lines respectively. Data was taken for four separate arrangements where the probe was positioned on a layer of myocardial tissue over a layer of fat. The thickness of the myocardial tissue layer was made approximately 10.0 mm in arrangement A, 4.0 mm in arrangement B and 1.5 mm C. The probe directly contacted the fat in arrangement D. The absorbance spectra were measured across a wavelength range of 1680 to 1780 nm. Peaks at around 1728 and 1766 (representing fat tissue) are shown that vary in intensity depending on the source-detector separation and the distance between the probe and fat tissue. Pursuant to various embodiments of the invention, similar data could be collected and modeled in order to prevent a puncturing by a probe into pericardial fat tissue from within myocardial tissue (and avoid causing serious harm to a patient).

In another example of pre-operational model data gathering, a probe in accordance with an embodiment of the invention could be placed in a blood medium at the appropriate temperature (i.e. 38° C.) with its position modified relative to targeted tissue (e.g. myocardium). The tissue types and their positions in relation to the probe would be known independently of data gathered from the probe to develop additional chemometric correlation models. This analysis would be useful for positioning and entry into the myocardium with the needle tip during actual operation.

Analysis that reflects fibrous or calcified tissue can often help identify the center of a myocardial infarct region, which can be surrounded by fibrous or calcified tissue. The degree of these indicators may also reflect levels of damage and general time periods during which the myocardial infarct lesions occurred (e.g. an acute lesion occurring less than 24 hours prior, a sub-acute myocardial infarct occurring less than one month prior, or chronic infarct occurring greater than one month prior). Data about tissue and blood, including oxygenation content and pH, is also obtainable using known spectroscopic analysis techniques and is useful for aiding diagnosis and for locating optimal tissue regions for delivering treatment. Analysis of oxygenation can be used in part to help assess whether myocardial tissue is damaged (e.g. necrotic) or normal. A study performed at the Oregon Medical Laser Center (Prahl, S., “Optical Absoroption of Hemoglobin”, Oregon Medical Laser Center (1999); retrieved from the Internet: <URL:http://omlc.ogi.edu/spectra/hemoglobin/index.html>, incorporated herein in its entirety by reference, for example, demonstrates characterizing spectra obtained from oxy and deoxy-hemoglobin. Collagen levels can also be measured to aid in the comparison of fibrous tissue associated with necrosis and normal (relatively collagen-free) tissue.

Embodiments of the invention also provide for enhanced tracking (real-time) the position of the distal end of the catheter as analysis is performed, providing enhanced calculations of the size, shape, and/or development of an infarcted area and transitions of tissue conditions therein. This information is highly useful for assessing the best area for applying treatment such as, for example, the affected areas surrounding an area of necrotic tissue. Based on present research, the most promising areas for applying treatment are regions within an infarct-affected area bordering completely necrotic tissue and tissue with some degree of viability, which could supply blood, oxygen, and nutrients for promoting advancement of healing or regeneration. A number of technologies are commercially available for enhanced real-time tracking of catheter movement, including, for example, fluoroscopy-based solutions, magnetic resonance imaging (MRI), image-guidance, rotary and linear translation, and precision encoders. Embodiments of the invention include features and materials (e.g. radiopaque materials) within the distal end of catheters detectable by, for example, a fluoroscope or MRI. For example, needle tip inserter 130 of FIGS. 2A-2B can include a highly radiopaque material such as, for example, platinum or gold detectable by a fluoroscope. In an embodiment of the invention, a controller (e.g. controller 50 of FIG. 1) can receive data from a tracking device (e.g. a fluoroscope) while the processor/analyzer receives simultaneously collected data from the probe end of the catheter so as to track and calculate the geometry, size, and position of targeted tissue within a patient.

A computer-aided output, such as visual representation, e.g. a graph or other output, or an audible presentation, can be provided to indicate to the operator the characterization of the myocardial tissue, including whether the myocardial area falls within one or more categories described above and/or to display the relative position of a suitable treatment area. The algorithms described above can be programmed into a central system processor and/or programmed or embedded into a separate processing device, depending on speed, cost, and other practical considerations.

Embodiments of the invention can also be adapted for studying the development of diseased tissues and assessing the effectiveness of treatment. After treatments are applied with use of the invention, for instance, the inventive catheter can be reinserted to assess the development and progress of the targeted areas. Information about the treatments and assessed tissue conditions can be recorded within the inventive system for purposes of determining future treatments and for conducting studies to optimize treatment plans in other patients.

It will be understood by those with knowledge in related fields that uses of alternate or varied forms or materials and modifications to the apparatus and methods disclosed are apparent. This disclosure is intended to cover these and other variations, uses, or other departures from the specific embodiments as come within the art to which the invention pertains.

What is claimed is:

1. An apparatus for probing and treating internal body organs comprising:
a catheter having a fiber probe arrangement and one or more treatment lumens; and
an analysis and treatment control system connected to said catheter, said analysis and control system programmed to characterize and locate damaged tissue via said fiber probe arrangement and to treat said damaged tissue with said one or more treatment lumens.

2. The apparatus of claim 1 further comprising a spectrometer connected to said fiber probe arrangement.

3. The apparatus of claim 1 wherein the distal end of said catheter comprises a needle tip inserter.

4. The apparatus of claim 3 wherein said needle tip inserter incorporates the probe ends of one or more fibers of said fiber probe arrangement and a dispersal port for said one or more treatment lumens.

5. The apparatus of claim 3 wherein said needle tip inserter is partially retractable within said catheter so as to ease the advancement of said catheter in a patient while permitting optical analysis.

6. The apparatus of claim 1 wherein the analysis and treatment control system is programmed to analyze spectroscopic data, the analysis of the spectroscopic data including distinguishing the types and conditions of tissue within and surrounding a patient's heart.

7. The apparatus of claim 6 wherein the spectroscopic data is selected according to predetermined wavelength bands that distinguish levels of at least one of particles, gas, and liquid contained in the tissue.

8. The apparatus of claim 6 wherein distinguishing the types and conditions of tissue within and surrounding a patient's heart includes characterizing and locating tissues associated with myocardial infarct.

9. The apparatus of claim 8 wherein the characterizing and locating tissues associated with myocardial infarct comprises identifying an area for treatment of myocardial infarction by locating and targeting an affected area surrounding a region of necrotic tissue.

10. The apparatus of claim 8 wherein characterizing and locating the tissues associated with myocardial infarct includes detecting levels of at least one of fibrosis, calcification, or oxygen content.

11. The apparatus of claim 10 wherein the analysis of said spectroscopic data includes chemometric analysis of said spectroscopic data in relation to previously obtained and stored spectroscopic data.

12. The apparatus of claim 11 wherein the chemometric analysis involves at least one technique, the at least one technique including Principle Component Analysis (PCA) with Mahalanobis Distance, PCA with K-nearest neighbor, PCA with Euclidean Distance, Partial Least Squares Discrimination Analysis, augmented Residuals, bootstrap error-adjusted single-sample technique, or Soft Independent Modeling of Class Analogy.

13. The apparatus of claim 10 wherein said analysis and control system is configured to perform spectroscopic scans across wavelengths within the range of approximately 300 to 2500 nanometers.

14. The apparatus of claim 6 wherein the analysis of the spectroscopic data includes estimating relative distances between a distal end of said fiber probe arrangement and tissue analyzed by said spectrometer.

15. The apparatus of claim 14 wherein estimating said relative distances includes comparing the magnitudes of spectroscopic absorbance peaks associated with tissue or blood with magnitudes similarly obtained from previously stored spectroscopic absorbance data.

16. The apparatus of claim 15 wherein estimating said relative distances includes comparing the magnitudes of the spectroscopic absorbance peaks obtained at different predetermined positions of said catheter relative to said tissue or blood.

17. The apparatus of claim 14 wherein estimating said relative distances includes comparing spectroscopic absorbance peaks associated with collection fibers having terminating ends separated longitudinally from each other at a predetermined distance.

18. The apparatus of claim 1 wherein said one or more treatment lumens comprises a conduit for delivering a fluid solution to damaged tissue.

19. The apparatus of claim 1 wherein said one or more treatment lumens comprises a conduit for delivering therapeutic laser energy.

20. The apparatus of claim 1 wherein said catheter further incorporates one or more sensors.

21. The apparatus of claim 20 wherein said one or more sensors include at least one temperature gauge, pH meter, oxygenation meter, or water content meter.

22. The apparatus of claim 1 wherein said catheter further includes a biopsy sampler.

23. The apparatus of claim 3 wherein the distal end of said catheter further comprises a guidewire branching from said catheter apart from said needle tip.

24. A catheter for probing and treating myocardial infarct, said catheter comprising:
a fiber probe arrangement;
one or more treatment lumens; and
a distal end having a needle injection inserter, said inserter integrating one or more fiber probe ends from said fiber probe arrangement and one or more delivery ports from said one or more treatment lumens.

25. The catheter of claim 24 further comprising an angle control wire for adjusting the angle of the distal end of said catheter.

26. The apparatus of claim 24 further comprising a gripping element about the proximate portion of said catheter, said gripping element having one or more control elements for controlling aspects of positioning said catheter or for delivering treatment.

27. A method for treating body tissue, said method comprising:
inserting into a patient a catheter integrated with a fiber optic analysis probe and a treatment delivery conduit;
characterizing and locating the body tissue to be treated with radiation delivered and collected through said fiber optic analysis probe;
positioning said catheter to deliver treatment with information obtained through said fiber optic analysis probe; and
delivering a treatment through said treatment delivery conduit.

28. The method of claim 27 wherein the body tissue to be treated is associated with myocardial infarct.

29. The method of claim 28 wherein locating the body tissue associated with myocardial infarct to be treated includes locating and targeting an affected area surrounding a region of necrotic tissue for the step of delivering a treatment through the treatment delivery conduit.

30. The method of claim 28 wherein characterizing and locating the body tissue associated with myocardial infarct to be treated comprises:
obtaining spectroscopic data from radiation delivered to and collected from said tissue to be treated via said fiber optic analysis probe; and
comparing said spectroscopic data with previously stored data characteristic of tissues within and around a patient's heart in order to identify the type of tissue being analyzed and to locate the position of said tissue being analyzed relative to said catheter.

31. The method of claim 30 wherein characterizing the tissue to be treated involves comparing levels of at least one of gases, fluids, and compounds within typical normal tissues as compared to at least one of gases, fluids, and compounds within tissues associated with myocardial infarct.

32. The method of claim 30 wherein obtaining spectroscopic data comprises at least one of the methods including diffuse-reflectance spectroscopy, fluorescence spectroscopy, Raman spectroscopy, scattering spectroscopy, optical coherence reflectometery, and optical coherence tomography.

33. The method of claim 30 wherein said at least one of gases, fluids, and compounds are selected from the group including collagen, calcium, oxygen, hemoglobin, and myoglobin.

34. The method of claim 30 wherein characterizing the tissue to be treated involves chemometric analysis selected from the group of techniques consisting of Principle Component Analysis (PCA) with Mahalanobis Distance, PCA with K-nearest neighbor, PCA with Euclidean Distance, Partial Least Squares Discrimination Analysis, augmented Residuals, bootstrap error-adjusted single-sample technique, and Soft Independent Modeling of Class Analogy.

35. The method of claim 30 wherein said radiation delivered and collected through said fiber optic probe is restricted to selectively narrow spans of wavelengths associated with identifying said tissues.

36. The method of claim 35 wherein the spectroscopic data is obtained from radiation spanning wavelengths between approximately 300 to 2500 nanometers.

37. The method of claim 3 wherein the spectroscopic data is selectively collected in sub-ranges of radiation spanning approximately 300 to 1375 nanometers, 1550 to 1850 nanometers, and 2100 to 2500 nanometers.

38. The method of claim 35 wherein radiation is delivered to tissue or blood at a narrow range including 380 nanometers and scanned across a narrow range including 320 nanometers in order to identify the presence of collagen.

39. The method of claim 30 wherein locating tissues in relation to said catheter includes pre-operative steps of analyzing and comparing the wavelengths and magnitudes of spectroscopic absorbance peaks associated with tissues and blood surrounding said tissues.

40. The method of claim 39 wherein the wavelengths and magnitudes of spectroscopic absorbance peaks associated with tissues and blood is compared with previously obtained and stored spectroscopic absorbance data associated with a catheter approaching similar tissues in a blood medium.

41. The method of claim 27 wherein the distal end of said catheter includes an inserter integrated with terminating ends of said fiber optic probe and delivery conduit, said inserter suitably sharp for perforating targeted tissue.

42. The method of claim 41 wherein, during positioning of said catheter for delivery of treatment, said integrated inserter remains at least partially retracted in said catheter prior to perforation into tissue targeted for treatment and said fiber optic probe is functional while said inserter is at least partially retracted.

43. The method of claim 42 wherein final positioning of said catheter for delivery of treatment includes extending said inserter out from the distal end of said catheter into the targeted tissue.

44. The method of claim 43 wherein, prior to and during extension of said inserter, a wall of myocardial tissue before which said inserter is positioned is concurrently analyzed and monitored to prevent complete perforation of said inserter through the entire wall of said myocardial tissue.

45. The method of claim 44 wherein the prevention of complete perforation includes monitoring the contents of tissue for a layer of pericardial fat positioned beyond said wall of myocardial tissue.

46. The method of claim 27 wherein delivering treatment through said treatment delivery conduit comprises the injection of therapeutic agents.

47. The method of claim 27 wherein delivering treatment through said treatment delivery conduit comprises the injection of chemical agents.

48. The method of claim 46 wherein delivering treatment through said treatment delivery conduit comprises the delivery of gene therapy agents.

49. The method of claim 46 wherein delivering treatment through said treatment delivery conduit comprises injecting stem cell therapy agents.

50. The method of claim 46 wherein delivering treatment through said treatment delivery conduit comprises injecting cytotherapy agents.

51. The method of claim 46 wherein one or more of a selection of therapy agents are chosen and delivered based on data collected during characterizing and locating the body tissue to be treated.

52. The method of claim 46 wherein the release of agents is monitored with said fiber optic probe and controlled using feedback from said monitoring.

53. The method of claim 27 wherein delivering treatment through said treatment delivery conduit comprises delivering therapeutic laser energy.

54. The method of claim 53 wherein delivering therapeutic laser energy comprises canalizing infarct tissue for purposes of revascularization.

55. The method of claim 27 wherein said catheter is introduced into said patient in accordance with a percutaneous transluminal angioplasty.

56. The method of claim 27 wherein said catheter is introduced into said patient in accordance with percutaneous endoventricular delivery.
